(19) United States
(12) Patent Application Publication
(10) Pub. No.: US 2023/0288179 A1
(43) Pub. Date: Sep. 14, 2023

(54) MAGNETISM MEASURING APPARATUS, MAGNETISM MEASUREMENT PROCESSING APPARATUS, AND METHOD FOR CONTROLLING MAGNETISM MEASUREMENT PROCESSING APPARATUS

(71) Applicant: Kazuma GOTO, Ishikawa (JP)

(72) Inventor: Kazuma GOTO, Ishikawa (JP)

(73) Assignee: Ricoh Company, Ltd., Tokyo (JP)

(21) Appl. No.: 18/167,952

(22) Filed: Feb. 13, 2023

(30) Foreign Application Priority Data
Mar. 9, 2022 (JP) 2022-036530

(51) Int. Cl. G01B 7/004; G01B 7/02

(52) U.S. Cl. CPC G01B 7/004; G01B 7/023

(57) ABSTRACT

Magnetism sensors having magnetism sensitivities with respect to respective single directions, and a coil configured to generate a magnetic field are used. A processor is configured to use some of the magnetism sensors to estimate a relative position between the coil and the some of the magnetism sensors based on magnetic fields; and avoid using, in calculation to estimate the relative distance, magnetism data measured by at least one of the magnetism sensors, the at least one of the magnetism sensors having an absolute value of a cosine similarity that is greater than or equal to a threshold, the cosine similarity being between a normal vector to the coil and a direction in which the at least one of the magnetism sensors has a magnetism sensitivity.

### CROSS-REFERENCE TO RELATED APPLICATIONS

The present application is based on and claims priority under 35 U.S.C. § 119 to Japanese Patent Application No. 2022-036530, filed Mar. 9, 2022. The contents of Japanese Patent Application No. 2022-036530 are incorporated herein by reference in their entirety.

### BACKGROUND OF THE INVENTION

### 1. Field of the Invention

The present invention relates to a magnetism measuring apparatus, a magnetism measurement processing apparatus, and a method for controlling a magnetism measurement processing apparatus.

### 2. Description of the Related Art

A magnetism measuring apparatus such as a magnetoencephalograph is an apparatus that measures a weak magnetic field generated through nerve activity with a superconducting quantum interference device (SQUID) sensor array or the like, and identifies a position of major nerve activity from a relationship between a magnetic field source and a magnetic field distribution. The magnetism measuring apparatus can measure a magnetic field, but cannot obtain an image of a neural activity measurement target such as a brain or a spinal cord.

Therefore, the portion of the human subject where the magnetic field is generated cannot be identified only by measuring the magnetic field. Therefore, by measuring a weak alternating current flowing through a marker coil disposed near the measurement target portion of the subject by a magnetism sensor of the magnetism measuring apparatus, it is possible to estimate the relative position between the magnetism sensor and the marker coil and identify the positional relationship between the measurement target portion of the subject and the measured magnetic field.

By identifying the positional relationship between the measurement target portion of the subject and the measured magnetic field using the marker coil, it is possible to display the distribution of the neural activity calculated from the biomagnetic field and the image of the subject in a superimposition manner (for example, see Non-Patent Document 1). In addition, in biomagnetic field measurement, it is possible to measure the amount of movement of the observed portion of the subject due to a movement of the body of the subject, or the like (see, for example, Non-Patent Document 2).

For example, there is known a method of calculating a reference point using a plurality of marker coils and estimating the position of the reference marker coils based on the calculated reference point (for example, see Patent Document 1). In addition, there is known a method in which magnetic fields are caused to be generated only from marker coils by causing currents of opposite phases to flow through spiral patterns of the marker coils formed on both surfaces of a flexible substrate, so as to prevent generation of magnetic fields due to currents flowing through the wires (for example, see Patent Document 2). In addition, there is known a method of dividing a magnetic field detection area with respect to magnetism sensors and thereby calculating changes in outputs of the magnetism sensors in accordance with the positional relationships between the outputs of the magnetism sensors and the magnetic field sources (for example, see Non-Patent Document 3).

### SUMMARY OF THE INVENTION

A magnetism measuring apparatus includes a plurality of magnetism sensors having magnetism sensitivities with respect to respective single directions; a coil configured to generate a magnetic field in response to supply of a current; and a processor configured to use a predetermined number of magnetism sensors from among the plurality of magnetism sensors to estimate a relative position between the coil and the predetermined number of magnetism sensors based on magnetic fields detected by the predetermined number of magnetism sensors. The processor is configured to avoid using, in calculation for estimating the relative position, magnetism data measured by at least one of the plurality of magnetism sensors, the at least one of the plurality of magnetism sensors having an absolute value of a cosine similarity that is greater than or equal to a threshold, the cosine similarity being between a normal vector to the coil and a direction in which the at least one of the plurality of magnetism sensors has a magnetism sensitivity.

Other objects, features, and advantages of the present invention will become more apparent from the following detailed description when read in conjunction with the accompanying drawings.

### BRIEF DESCRIPTION OF THE DRAWINGS

FIGS. 1A and 1B are block diagrams depicting an example of a configuration of a magnetism measuring apparatus according to a first embodiment;

FIG. 2 is a diagram depicting an example of a structure of a marker coil of FIGS. 1A and 1B;

FIG. 3 is an explanatory diagram depicting an example of conditions for calculating a magnetic field detected by a magnetism sensor based on a magnetic field generated by a magnetic dipole;

FIG. 4 is an explanatory diagram depicting an example of a simulation result when a magnetic field detected by a magnetism sensor is calculated under the conditions depicted in FIG. 3;

FIG. 5 is an explanatory diagram depicting an example of conditions for calculating a magnetic field detected by a magnetism sensor in a case where a relationship between an orientation of a magnetic dipole and an orientation of a detection coil is considered;

FIG. 6 is an explanatory diagram depicting an example of a simulation result when a magnetic field detected by a magnetism sensor is calculated under the conditions depicted in FIG. 5;

FIG. 7 is a diagram depicting an example of simulation conditions for estimating a position of a marker coil by a magnetism measuring apparatus different from the magnetism measuring apparatus of FIGS. 1A and 1B;

FIG. 8 is a diagram depicting an example of an arrangement of magnetism sensors and a marker coil under the simulation conditions depicted in FIG. 7;

FIG. 9 is an explanatory diagram depicting an example of estimation error of the position of the marker coil obtained using the simulation conditions depicted in FIGS. 7 and 8;

FIGS. 10A and 10B are block diagrams depicting an example of a configuration of a magnetism measuring apparatus according to a second embodiment;

FIG. 11 is a diagram depicting an example of simulation conditions for estimating the position of the marker coil by a magnetism measuring apparatus different from the magnetism measuring apparatus of FIGS. 10A and 10B;

FIG. 12 is an explanatory diagram depicting an example of estimation error of the position of the marker coil obtained using the simulation conditions depicted in FIG. 11; and

FIGS. 13A and 13B are explanatory diagrams depicting an example of a positional relationship between each magnetism sensor of a magnetism sensor array and a marker coil in a magnetism measuring apparatus according to a third embodiment.

### DESCRIPTION OF THE EMBODIMENTS

In the related art described above, when the magnetic fields generated from the marker coils are affected by noise for example, the accuracy of the estimated positions of the marker coils may be reduced.

An object of embodiments is to improve estimation accuracy of a position of a coil used as a marker in a magnetism measuring apparatus.

According to the embodiments that will now be described, it is possible to improve marker coil position estimation accuracy in a magnetism measuring apparatus.

Hereinafter, the embodiments will be described with reference to the drawings. In the drawings, the same elements are provided with the same reference numerals, and the duplicate description may be omitted.

### First Embodiment

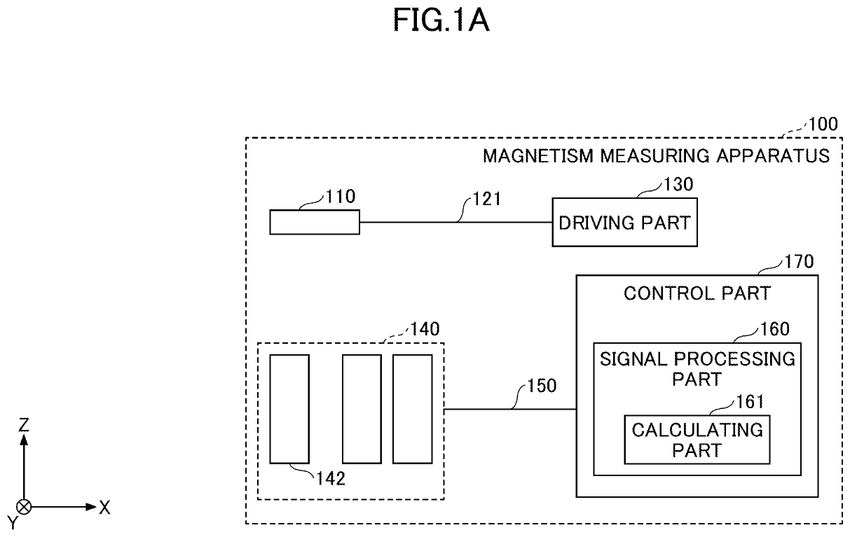

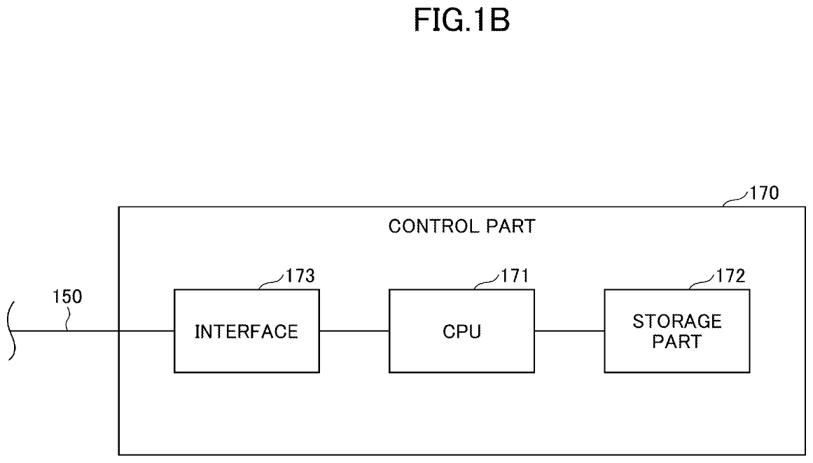

FIGS. 1A and 1B are block diagrams depicting an example of a configuration of a magnetism measuring apparatus according to a first embodiment. The magnetism measuring apparatus 100 depicted in FIG. 1A includes a marker coil 110 used as a marker, a driving part 130, and lead wires 121 connecting between the marker coil 110 and the driving part 130. The magnetism measuring apparatus 100 includes a magnetism sensor array 140 including a plurality of magnetism sensors 142, a control part 170 including a signal processing part 160, and wires 150 connecting between the magnetism sensors 142 and the signal processing part 160.

As depicted in FIG. 1B, the functions of the control part 170, including the signal processing part 160, may be implemented by a central processing unit (CPU) 171 (an example of a processor), a storage part 172, and an interface 173. The CPU 171 executes a program stored in the storage part 172 to perform the processing that is performed by the signal processing part 160. The interface 173 is an interface for the CPU 171 to receive signals representing magnetism detected by the magnetism sensor array 140 via the wires 150. The storage part 172 may include a hard disk drive, a random access memory (RAM), a read-only memory (ROM), and so forth. The program may be previously stored in a hard disk of the hard disk drive or the ROM, or may be provided from an external server or the like via a communication network such as the Internet.

The magnetism measuring apparatus 100 may be applied as a biomagnetism measuring apparatus such as a magnetospinograph (MSG), a magnetomyograph (MMG), a magnetoencephalograph (MEG), a magnetocardiograph (MCG), or the like. In addition, the magnetism measuring apparatus 100 may be applied as a magnetic motion capture apparatus, a magnetic tracking apparatus, or the like, detecting a position or a motion of an object to which a coil is attached by detecting a magnetic field generated from the coil. That is, the application range of the magnetism measuring apparatus 100 is not limited to apparatuses that are used for living bodies.

The driving part 130 supplies a current to the marker coil 110 via the lead wires 121, and causes the marker coil 110 to generate a magnetic field. The marker coil 110 may have any shape as long as it generates a magnetic field according to the Biot-Savart law when being supplied with a current. It is preferable that the driving part 130 can supply a sinusoidal current to the marker coil 110 in order that a magnetic field generated in response to a current being supplied to the marker coil 110 can be distinguished from external magnetic field noise or the like.

The lead wires 121 are covered with an insulating material such as polyimide. The lead wires 121 are wires disposed in such a manner that current vectors of currents flowing through the conductors of the lead wires 121 are parallel to the X-axis in FIG. 1A. Note that the lead wires 121 may be disposed in such a manner as to be parallel to a direction different from the X-axis as long as the orientation of the lead wires is known. The lead wires 121 may be disposed at an any orientation, at a position sufficiently distant from the magnetism sensors 142 such that the magnetism sensors 142 cannot detect the magnetic fields generated from the lead wires 121.

Each magnetism sensor 142 detects a magnetic field generated when a current flows through the marker coil 110, and transmits a signal indicating the detected magnetic field to the signal processing part 160 via the wires 150. For example, the magnetism sensors 142 have magnetism sensitivities in a Z-axis direction (single direction) which is a direction toward the marker coil 110. That is, the magnetism sensors 142 have directivities in the direction in which the magnetism sensors 142 readily detect the target magnetism. For example, SQUID sensors are used as the magnetism sensors 142. The SQUID sensors require cooling mechanisms (not depicted) for maintaining their superconducting states. In order to detect magnetic fluxes, the SQUID sensors have, for example, detection coils each with a radius of 20 mm. Each of the magnetism sensors 142 has a sensitivity in the direction normal to the detection coil. It is noted that a vector normal to a plane corresponding to the annular shape of the detection coil is referred to as a normal vector to the detection coil.

The control part 170 controls operation of each magnetism sensor 142 (for example, turning on and off the detecting operations of the magnetism sensors 142 with respect to magnetic fields). The signal processing part 160 includes a calculating part 161 that performs data processing on a signal received from each magnetism sensor 142. The signal processing part 160 includes a flux locked loop (FLL) circuit and an analog-to-digital converting circuit (not depicted). The signal processing part 160 converts a signal transmitted from each magnetism sensor 142 via the wires 150 into a digital signal. The signal processing part 160 is an example of a magnetism measurement processing apparatus and includes the calculating part 161 that processes magnetism data measured by the plurality of magnetism sensors 142.

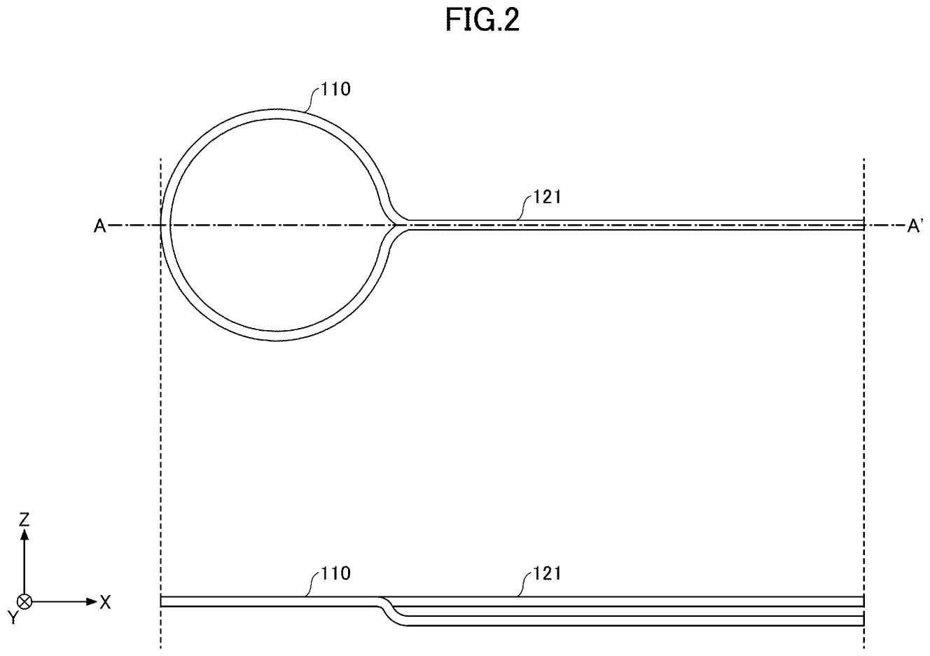

FIG. 2 is a diagram depicting an example of a structure of the marker coil 110 of FIG. 1A. The upper part of FIG. 2 depicts a plan view of the marker coil 110, and the lower part of FIG. 2 depicts a cross-sectional view, taken along a line A-A′, of the plan view. The marker coil 110 is disposed in such a manner that a current flows on an X-Y plane. Therefore, when a current flows through the marker coil 110, a magnetic field is generated in a Z-axis direction. It is noted that a vector normal to a plane corresponding to the annular shape of the marker coil 110 is referred to as a normal vector to the marker coil 110. In FIG. 2, the normal vector to the marker coil 110 is parallel to the Z-axis. The lead wires 121 are preferably twisted wires so as not to generate magnetic field noise when currents pass through the lead wires 121.

The calculating part 161 depicted in FIG. 1A assumes the marker coil 110 as a magnetic dipole and solves an inverse problem using an optimization method based on the amplitudes and phases of magnetic field waveforms detected by the magnetism sensors as described in Non-Patent Document 2. Thus, the calculating part 161 performs a process of deriving the position of the marker coil 110 to estimate the position of the marker coil 110 relative to the positions of a predetermined number of magnetism sensors from among the magnetism sensors 142 (that is, a relative position between the marker coil 110 and the predetermined number of magnetism sensors from among the magnetism sensors 142).

Note that, according to the first embodiment, as will be described later, the magnetism data measured by certain one or more of the magnetism sensors are avoided from being used for calculation to estimate the position of the marker coil 110 in order to improve the accuracy of estimating the position of the marker coil 110. That is the reason why, as described above, the calculating part 161 performs a process of deriving the position of the marker coil 110 to estimate the position of the marker coil 110 relative to the positions of a predetermined number of magnetism sensors from among the magnetism sensors 142 (that is, a relative position between the marker coil 110 and the predetermined number of magnetism sensors from among the magnetism sensors 142). The predetermined number of magnetism sensors from among the magnetism sensors 142 are magnetism sensors whose magnetism data is used for calculation to estimate the position of the marker coil 110; and do not include the above-mentioned certain one or more of the magnetism sensors whose magnetism data is avoided from being used for calculation to estimate the position of the marker coil 110.

The calculating part 161 calculates magnetic fields that would be detected by the magnetism sensors using the following equation (1) depicted in Non-Patent Document 2, and, for this purpose, uses the position vectors with respect to the single central points of the detection coils. The equation (1) is applicable to the magnetism measuring apparatus including the plurality of magnetism sensors 142.

$${B}_{i} = {S}_{i} \cdot \frac{1}{4 π} \{ \frac{1}{{\vert {r}_{i} \vert}^{3}} ( \frac{3 {r}_{i} \cdot m}{{\vert {r}_{i} \vert}^{2}} {r}_{i} - m ) \} \qquad ( 1 )$$

In the equation (1), the symbol m represents a magnetic dipole moment (the marker coil 110). The symbol Sᵢ represents a unit vector indicating the orientation of the i-th magnetism sensor 142. The symbol r, represents a vector originating from the magnetic dipole and directed to the i-th magnetism sensor 142.

The calculating part 161 calculates a signal Bi for when a magnetic field generated by the magnetic dipole is detected at one point, as a representative, of the detection coil included in each of the magnetism sensors 142. Actually, the magnetic field generated by the magnetic dipole has a distribution in the plane of the detection coil, and as a result, when the signal Bi is calculated by the calculating part 161 with the representative one point of the detection coil, an error is generated due to the distribution of the magnetic field in the detection coil

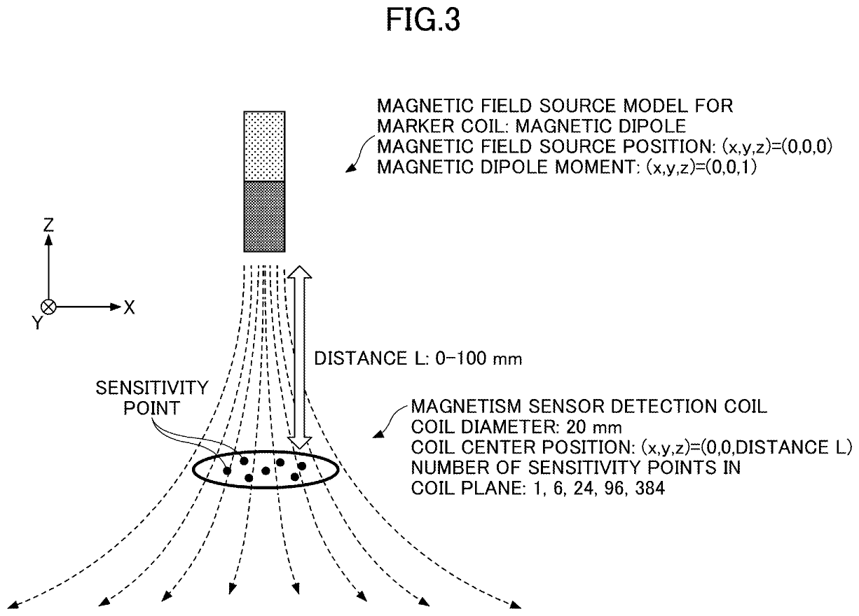

FIG. 3 is an explanatory diagram depicting an example of conditions for calculating the magnetic field that would be detected by the magnetism sensor 142 based on the magnetic field generated by the magnetic dipole. A model of the magnetic field source (marker coil 110) used in the calculation is the magnetic dipole. The position of the magnetic field source model is set to (x, y, z)=(0, 0, 0) and the end point of the magnetic dipole moment is set to (x, y, z)=(0, 0, 1).

Each of the detection coils of the magnetism sensors 142 used for the calculation is set to have a diameter of 20 mm, a center position of the detection coil is set to (x, y, z)=(0, 0, distance L), and the number of sensitivity points in the plane of the detection coil (“coil plane” in FIG. 3) is set to each of 1, 6, 24, 96, and 384. The distance L is a distance from the magnetic dipole to the detection coil and is set between the 0 mm and the 100 mm. For example, for the number of sensitivity points=“1”, the sensitivity point is set at a position that is in the Z-axis direction with respect to the magnetic dipole in the plane of the detection coil. For the number of sensitivity points=“6”, the sensitivity points are set at the respective apexes of a regular hexagon with its center at the position that is in the Z-axis direction with respect to the magnetic dipole in the plane of the detection coil.

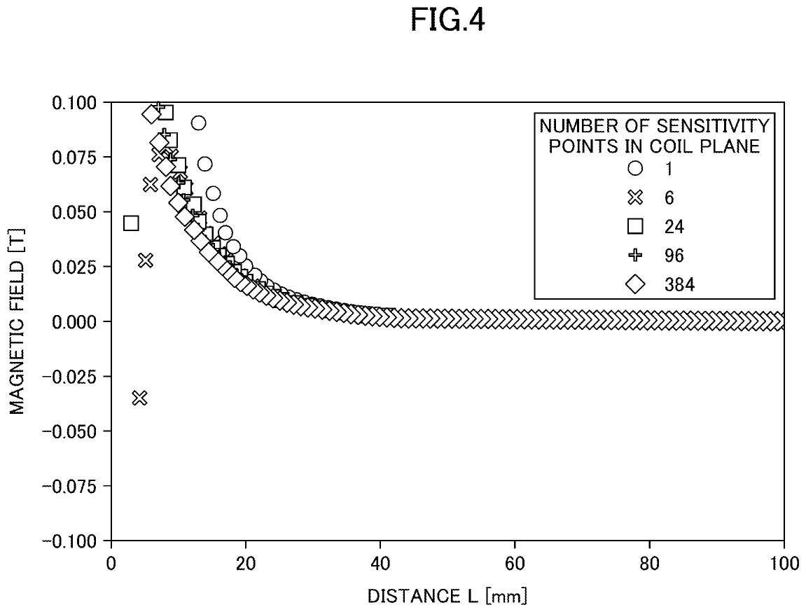

For example, the distance L is indicated by the Z component of the coil center position of the detection coil of the magnetism sensor 142. In the simulation depicted in FIGS. 4-6, as described above, the position of the magnetic field source model was set to the coordinate system origin (x, y, z)=(0, 0, 0), and the coil center position of the detection coil of the magnetism sensor 142 was set to (x, y, z)=(0, 0, L). Therefore, when the position vector of the magnetic field source model is indicated as pm and the position vector of the coil center of the detection coil of the magnetism sensor 142 is indicated as pₛ, the absolute value of the difference between these position vectors is obtained by the following equation (2):

|pₘ−pₛ|=\sqrt{(0−0)²+(0−0)²+(0−L)²}=L (2)

The equation (2) indicates that the distance L is the absolute value of the difference between these position vectors and also is the Z component of the coil center position of the detection coil of the magnetism sensor 142.

FIG. 4 is an explanatory diagram depicting an example of a simulation result obtained from the magnetic field that would be detected by the magnetism sensor 142 being calculated under the conditions depicted in FIG. 3. From the simulation result, it can be seen that, as the distance L between the magnetic dipole and the detection coil decreases, the calculation result for the case where the number of sensitivity points in the plane of the detection coil (“coil plane” in FIG. 4) is “1” and the calculation result for the case where the number of sensitivity points in the plane of the detection coil is more than “1” tend to diverge from one another. In other words, it can be seen that, as the distance L between the magnetic dipole and the detection coil decreases, the influence of calculation error due to the magnetic field distribution in the plane of the detection coil becomes more significant. It is noted that, in a case where the number of sensitivity points is more than “1” in the simulation, the magnetic field was obtained through averaging the magnetic fields calculated for the respective sensitivity points.

From the example depicted in FIG. 3, it can be seen that, for a case where the number of sensitivity points is “1”, the sensitivity point is located at a position to face the center of the marker coil 110 with respect to the X-Y plane, and in this case, there is a possibility that the calculation error becomes greater than the case where the number of the sensitivity points is more than two. Therefore, when a magnetic field is calculated using a single sensitivity point, it is preferable to avoid using magnetism data detected by the magnetism sensor 142 having a sensitivity point at a position to face the center of the marker coil 110 with respect to the X-Y plane in calculation for estimating the position of the marker coil 110. It is noted that although FIG. 4 depicts the calculation results in a manner of focusing on the distance L between the magnetic dipole and the detection coil, the magnetic field distribution in the plane of the detection coil varies also depending on a combination of the orientation of the magnetic dipole and the orientation of the detection coil.

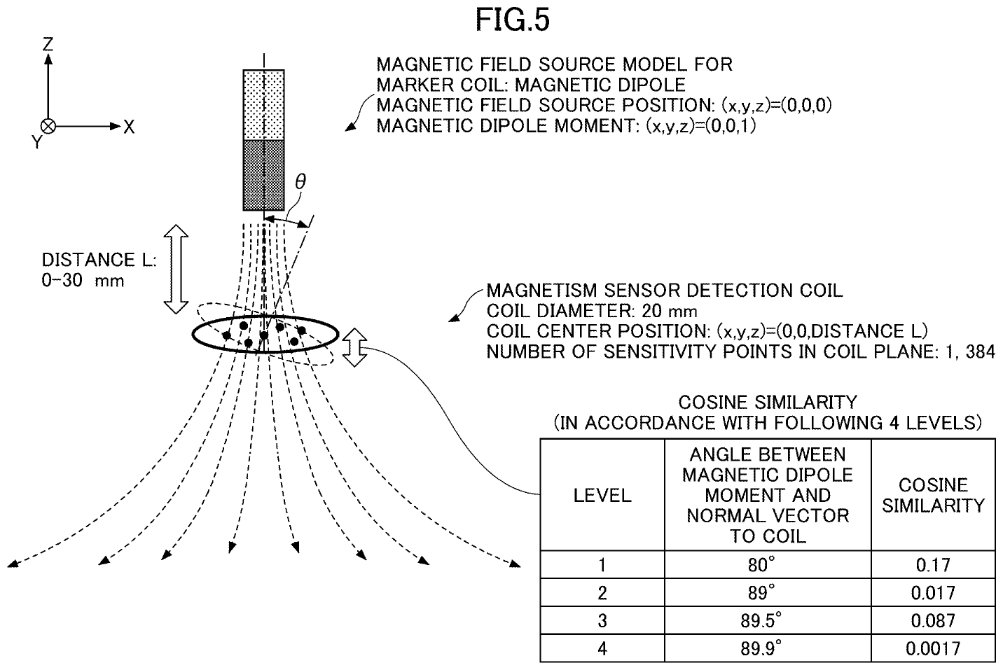

FIG. 5 is an explanatory diagram depicting an example of conditions for calculating a magnetic field that would be detected by a magnetism sensor 142 in a case where the relationship between the orientation of the magnetic dipole and the orientation of the detection coil is considered. What is different from the example of FIG. 3 is the following three points: (1) the distance L from the magnetic dipole to the detection coil was set to 0 mm to 30 mm; (2) the number of sensitivity points in the plane of the detection coil (“coil plane” in FIG. 5) was set to 1 and 384; and (3) the angle formed by the magnetic dipole moment and the normal vector to the detection coil was set to each of 80°, 89°, 89.5°, and 89.9°.

In the above-described calculation of the magnetic field that would be detected by the magnetism sensor 142, the magnetic dipole moment was regarded as a constant. However, the magnetic dipole moment actually can have any quantity. Therefore, in the following description, cosine similarity is used as an index indicating a relationship between the magnetic dipole moment and the normal vector to the detection coil.

The cosine similarity is calculated by the equation (3) below. The greater the cosine similarity is, the smaller the angle θ between the normal vector to the marker coil 110 and the normal vector to the detection coil is; and the smaller the cosine similarity is, the greater the angle θ between the normal vector to the marker coil 110 and the normal vector to the detection coil is. θ depicted in FIG. 5 is an example of the angle formed by the normal vector to the marker coil 110 and the normal vector to the detection coil. In the equation (3), the symbol m represents the magnetic dipole moment, and the symbol s represents the normal vector to the detection coil.

$$cosine similarity = \frac{m \cdot s}{\vert m \vert \vert s \vert} \qquad ( 3 )$$

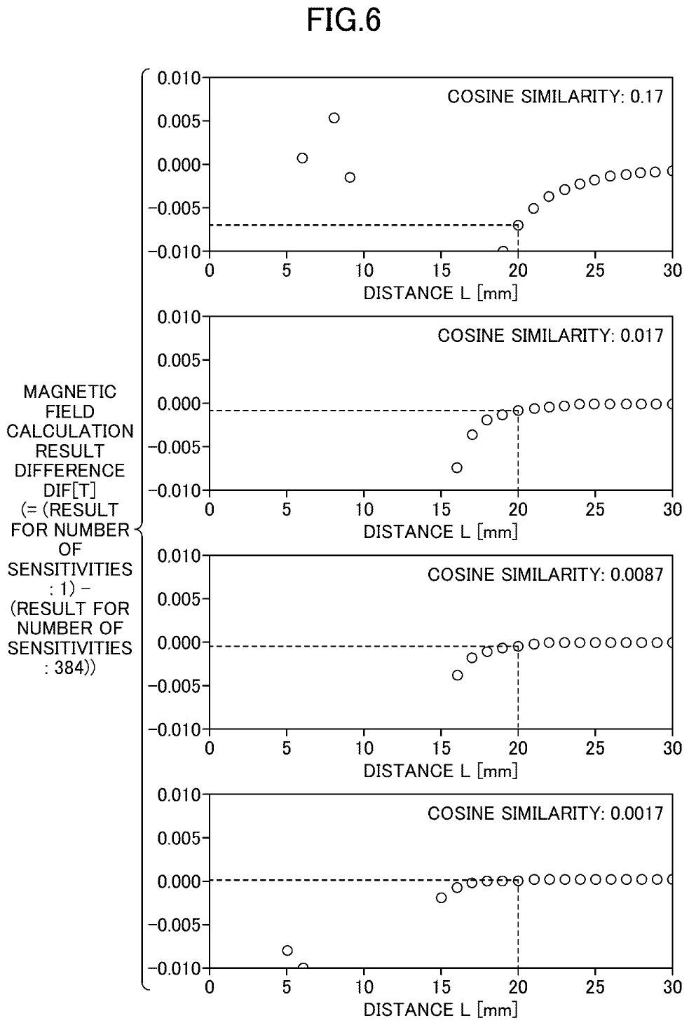

FIG. 6 is an explanatory diagram depicting an example of simulation results obtained when the magnetic field that would be detected by the magnetism sensor 142 was calculated under the conditions depicted in FIG. 5. From the example depicted in FIG. 4, it can be seen that the distance dependence of the calculation results of the magnetic fields in the case of the number of sensitivity points=“1” is characteristic. Therefore, in the example of FIG. 6, the differences DIF between the calculation results of the magnetic fields in the case of the number of sensitivity points=“1” and the calculation results of the magnetic fields in the case of the number of sensitivity points=“384” are used as indexes of the magnitudes of the calculation error in the case of the sensitivity point=“1”. It is noted that, in a case where the number of sensitivity points is more than “1” in the simulation, the magnetic field was obtained through averaging the magnetic fields calculated for the respective sensitivity points.

The four graphs depicted in FIG. 6 depict calculation results obtained when the cosine similarities (that are the absolute values, the same manner applying hereinafter) were “0.17”, “0.017”, “0.0087”, and “0.0017” from the top. The vertical axis of each graph indicates the differences between the magnetic fields calculated when the number of sensitivity points=“1” and the magnetic fields calculated when the number of sensitivity points=“384”. The horizontal axis of each graph indicates the distances L between the magnetic dipole and the detection coil. For example, with regard to the differences DIF in the case of the distance L=20 mm, it can be seen that the absolute value of the difference DIF is smaller as the cosine similarity is smaller.

That is, it can be seen that, even when the distance L between the marker coil 110 as the magnetic field source and the detection coil of the magnetism sensor 142 is small, the calculation error can be reduced if the cosine similarity is small. Therefore, by setting the positional relationship between the magnetic dipole and the magnetism sensor 142 such that the calculation error caused by the magnetic field distribution in the plane of the detection coil can be reduced, the position of the marker coil 110 can be estimated with higher accuracy.

In the first embodiment, the calculating part 161 avoids using, in the calculation for estimating the position of the marker coil 110, magnetism data measured by a magnetism sensor 142 having a magnetism detection direction in which the absolute value of the cosine similarity with respect to the normal vector to the marker coil 110 is “0.01” or more and “1” or less, for example. “0.01” is an example of a first threshold. As a result, it is possible to improve the accuracy of the position of the marker coil 110 estimated based on the magnetism data measured by the magnetism sensors 142. Instead of avoiding using the magnetism data in the calculation for estimating the position of the marker coil 110, the control part 170 depicted in FIG. 1 may stop the magnetic field detecting operation performed by the magnetism sensor 142 that detects the magnetism data of which the use is to be avoided (that is, may deactivate the corresponding magnetism sensor 142).

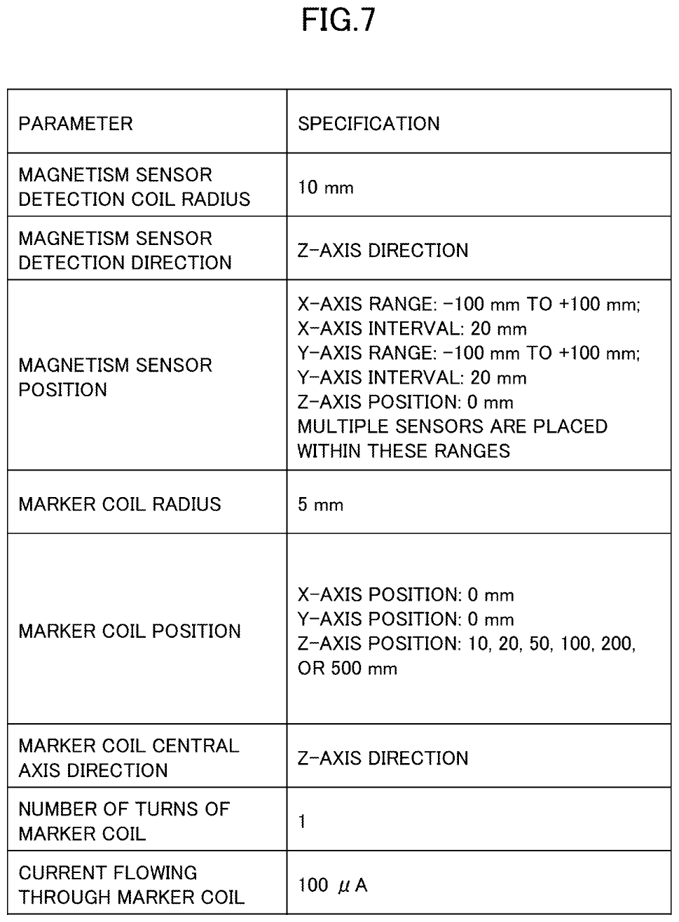

FIG. 7 is a diagram depicting an example of simulation conditions for estimating the position of the marker coil 110 by a magnetism measuring apparatus different from the magnetism measuring apparatus 100 of FIGS. 1A and 1B. The simulation was performed on the assumption that a plurality of magnetism sensors 142 having the detection directions with respect to magnetic fields (that is, the directivities of the detection sensitivities with respect to magnetism) that were parallel to the Z-axis direction were arranged at 20 mm intervals on an X-Y plane. The Z-axis position of the marker coil 110, which is indicated as the distance L from the magnetism sensor array 140 to the marker coil 110, was changed to six levels of 10 mm, 20 mm, 50 mm, 100 mm, 200 mm, and 500 mm. With respect to each of these positions of the marker coil 110, the magnetic field that would be generated according to the Biot-Savart law was calculated, instead of being actually measured by the magnetism measuring apparatus 100, and the calculated magnetic field was used to estimate the position of the marker coil 110.

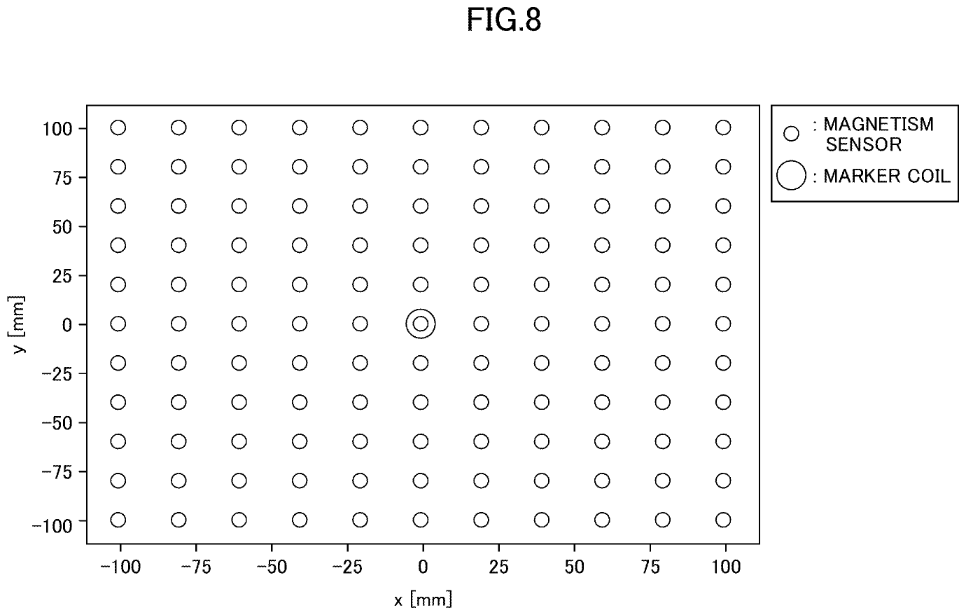

FIG. 8 is a diagram depicting an example of the arrangement of the magnetism sensors 142 and the marker coil 110 under the simulation conditions depicted in FIG. 7. For example, the magnetism sensors 142 were arranged at equal intervals in the range of 200 mm in each of the Y-axis direction and the X-axis direction. The number of the arranged magnetism sensors 142 was 11 in each of the Y-axis direction and the X-axis direction of FIG. 1. The marker coil 110 was placed at the center (x=0 mm, y=0 mm) of the area where the magnetism sensors 142 were arranged as depicted in FIG. 8.

The estimation errors of the position of the marker coil 110 in the X-axis direction, the Y-axis direction, and the Z-axis direction of the marker coil 110 were calculated using the following equations (4), (5) and (6), respectively. The three-dimensional estimation error of the position of the marker coil 110 was calculated by the equation (7) below using the respective calculation results of the equations (4), (5), and (6). In the equations (4), (5), and (6), “estimated position” and “actual position” indicate the estimated position of the marker coil 110 calculated and the actual position of the marker coil 110, respectively. Hereinafter, the estimation error of the position of the marker coil 110 may also be simply referred to as a “position estimation error”.

X-axis direction estimation error eₓ=(X-axis direction estimated position)−(X-axis direction actual position) (4)

Y-axis direction estimation error e_{y}=(Y-axis direction estimated position)−(Y-axis direction actual position) (5)

Z-axis direction estimation error e_{z}=(Z-axis direction estimated position)−(Z-axis direction actual position) (6)

position estimation error=\sqrt{eₓ²+e_{y}²+e_{z}²} (7)

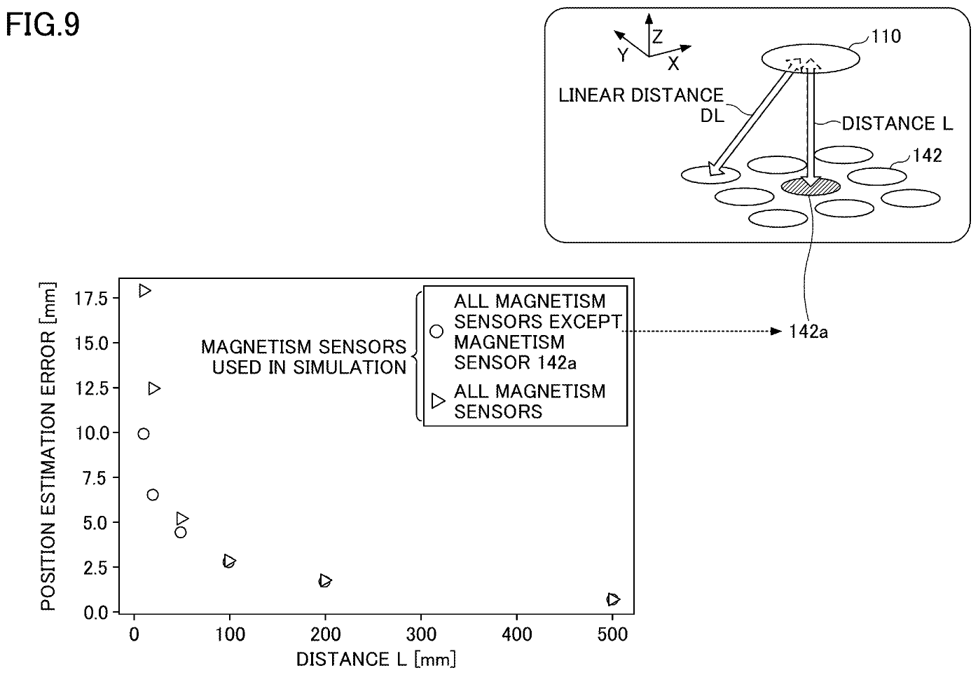

With regard to the plurality of magnetism sensors depicted in FIG. 8, the magnetism sensors whose normal vectors were parallel to each other had the same cosine similarities. In addition, a linear distance (“DL” depicted in FIG. 9) to the marker coil increased as the magnetism sensor was farther away from the marker coil in the X-Y plan view. Therefore, as the magnetism sensor was farther away from the marker coil in the X-Y plan view, the calculation error of the magnetic field generated from the marker coil was smaller, and the position estimation error of the marker coil was smaller.

FIG. 9 is an explanatory diagram depicting an example of the estimation error of the position of the marker coil 110 obtained using the simulation conditions depicted in FIGS. 7 and 8. In the graph of FIG. 9, the horizontal axis represents the distance L between the X-Y plane on which the magnetism sensors 142 are disposed and the marker coil 110. The vertical axis represents the position estimation error calculated using the equations (4) to (7). The distance L used in the simulation was a distance between the plane of the marker coil 110 (the plane perpendicular to the normal vector to the marker coil 110) and a surface on which the front ends of the plurality of magnetism sensors 142 of the magnetism sensor array 140 were arranged. On the other hand, when the magnetic fields were calculated from the positional relationships between the marker coil 110 and the magnetism sensors 142, the linear distances that were the distances between the position of the marker coil 110 and the center positions of the detection coils of the magnetism sensors 142 were used. The distances between the position of the marker coil 110 and the center positions of the detection coils of the magnetism sensors 142 are referred to as “linear distances DL”.

The circles in the graph represent the position estimation errors calculated for the case where all of the plurality of magnetism sensors 142, except the magnetism sensor 142a that faces the marker coil 110 along the normal vector to the marker coil 110, were used. For example, according to the example of FIG. 8, the position estimation errors were calculated for the case where the 120 magnetism sensors 142 (11×11−1=120), not including the central magnetism sensor (142a) that faces the marker coil 110 in the Z-axis direction, were used. The triangles in the graph represent the position estimation errors calculated for the case where all the magnetism sensors 142 were used. For example, in FIG. 8, the position estimation errors were calculated for the case where all of the 121 magnetism sensors 142 (11×11=121) were used.

According to the graph depicting the simulation result of FIG. 9, the position estimation error tends to decrease as the distance L between the X-Y plane on which the magnetism sensors 142 were disposed and the marker coil 110 increases. In addition, the advantageous effect indicated by a difference between the position estimation error for the case where all the magnetism sensors 142 were used and the position estimation error for the case where the magnetism sensor 142a was avoided being used tends to increase as the distance L between the X-Y plane on which the magnetism sensors 142 were disposed and the marker coil 110 decreases. That is, it can be seen that, the smaller the distance L between the X-Y plane on which the magnetism sensors 142 were disposed and the marker coil 110 is, the more remarkable the advantageous effect of reducing the position estimation error obtained by avoiding using the magnetic datum measured by the magnetism sensor 142a is. Note that the distance L (between the X-Y plane on which the magnetism sensors 142 were disposed and the marker coil 110) used in the simulation is different from the linear distance DL. For example, in the example of FIG. 9, the linear distance DL between any of the magnetism sensors 142 except the magnetism sensor 142a and the marker coil 110 is larger than the distance L (between the X-Y plane on which the magnetism sensors 142 were disposed and the marker coil 110). However, the linear distance DL between the magnetism sensor 142a and the marker coil 110 is equal to the distance L (between the X-Y plane on which the magnetism sensors 142 were disposed and the marker coil 110).

Therefore, for example, in the case where the distance L (between the X-Y plane on which the magnetism sensors 142 were disposed and the marker coil 110) is 50 mm, the calculating part 161 may avoid using, in the calculation for estimating the position of the marker coil 110, the magnetism data measured by the magnetism sensors 142 whose linear distances DL to the marker coil 110 are each equal to or less than 50 mm. The 50 mm is an example of a first distance. As a result, it is possible to improve the accuracy of the position of the marker coil 110 estimated based on the magnetism data measured by the magnetism sensors 142. The calculating part 161 may avoid using, in the calculation for estimating the position of the marker coil 110, the magnetism data measured by the magnetism sensors 142 whose distances from the marker coil 110 are each equal to or less than the first distance and the magnetism data measured by the magnetism sensors 142 whose cosine similarities with respect to the normal vector to the marker coil 110 are each equal to or greater than the first threshold.

As described above, in the first embodiment, the calculating part 161 avoids using, in the calculation for estimating the position of the marker coil 110, the magnetism data measured by the magnetism sensors 142 having the magnetism detection directions with which the cosine similarities with respect to the normal vector to the marker coil are each equal to or greater than the first threshold. Alternatively, the calculating part 161 avoids using, in the calculation for estimating the position of the marker coil 110, the magnetism data measured by the magnetism sensors 142 whose distances to the marker coil 110 are each equal to or less than the first distance. Furthermore, the calculating part 161 avoids using, in the calculation for estimating the position of the marker coil 110, the magnetism data measured by the magnetism sensors 142 whose distances from the marker coil 110 are each equal to or less than the first distance and the magnetism sensors 142 whose cosine similarities with respect to the normal vector to the marker coil 110 are each equal to or greater than the first threshold.

Thus, it is possible to improve the accuracy of the position of the marker coil 110 estimated based on the magnetism data measured by the magnetism sensors 142. As a result, when the magnetic field is measured by the magnetism measuring apparatus 100, the position of the marker coil 110 can be accurately estimated, and the magnetic field can be accurately measured by the magnetism measuring apparatus 100.

The magnetism sensor array 140 depicted in FIG. 1 may include a plurality of magnetism sensors 142 that include magnetism sensors having the magnetism detection sensitivities in the X-axis direction, magnetism sensors having the magnetism detection sensitivities in the Y-axis direction, and magnetism sensors having the magnetism detection sensitivities in the Z-axis direction. In this case, for example, in the arrangement of the magnetism sensors depicted in FIG. 8, the magnetism sensors having the magnetism detection sensitivities in the X-axis direction, the magnetism sensors having the magnetism detection sensitivities in the Y-axis direction, and the magnetism sensors having the magnetism detection sensitivities in the Z-axis direction are alternately arranged.

That is, the magnetism sensor array 141 may include at least one magnetism sensor 142 whose magnetism detection direction is parallel to the normal vector to the marker coil 110 and at least one magnetism sensor 142 whose magnetism detection direction is not parallel to the normal vector to the marker coil 110. In this case, for example, the calculating part 161 avoids using magnetism data measured by at least one magnetism sensor 142 disposed at a position to face the marker coil 110 and having the detection sensitivity in the Z-axis direction in calculation for estimating the position of the marker coil 110.

The magnetism measuring apparatus 100 may be a biomagnetism measuring apparatus such as a magnetoencephalograph. In this case, the plurality of magnetism sensors 142 mounted on the magnetoencephalograph are arranged at mutually different angles to fit a curved surface of a head of a subject. As a result, the magnetism detection directions of the plurality of magnetism sensors 142 are shifted little by little among the magnetism sensors 142. In this case, the calculating part 161 executes calculation for estimating the position of the marker coil 110 without using the magnetism data obtained by the magnetism sensors 142 whose magnetism detection directions are each in the same direction as the normal to the marker coil 110. Thus, the magnetoencephalograph can measure a biomagnetic field with high accuracy.

### Second Embodiment

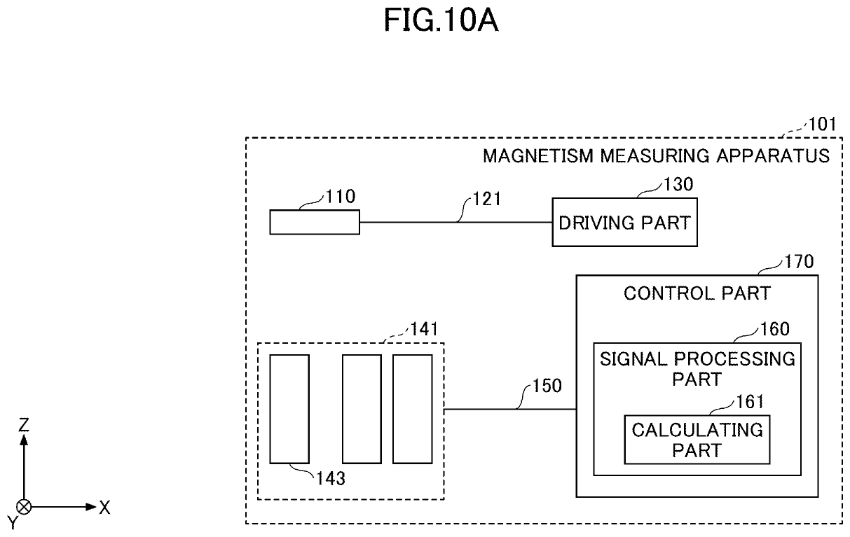

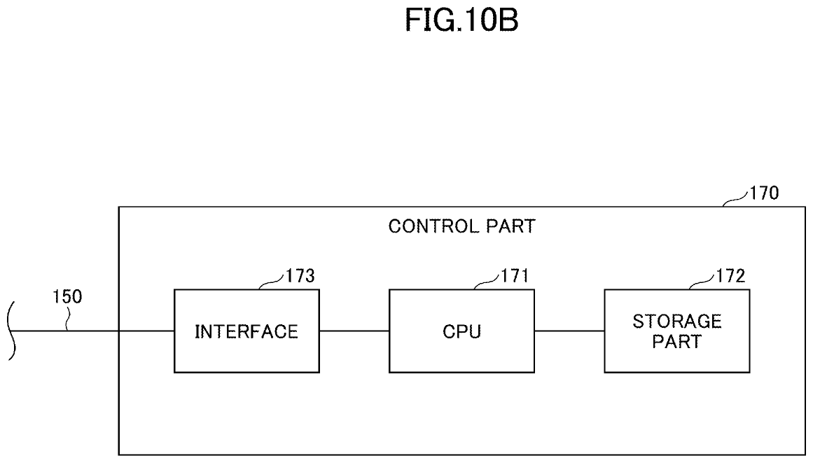

FIGS. 10A and 10B are block diagrams depicting an example of a configuration of a magnetism measuring apparatus according to a second embodiment. Elements similar to the elements depicted in FIG. 1 are indicated by the same reference numerals and will not be described in detail. The magnetism measuring apparatus 101 of the second embodiment includes a magnetism sensor array 141 instead of the magnetism sensor array 140 of FIG. 1. The magnetism sensor array 141 includes a plurality of magnetism sensors 143 instead of the plurality of magnetism sensors 142 of FIG. 1. For example, the number and arrangement of the magnetism sensors 143 are the same as the number and arrangement of the magnetism sensors 142 depicted in FIG. 8. The configuration of the magnetism measuring apparatus 101 is the same as the configuration depicted in FIG. 1 except for the magnetism sensors 143.

Each of the magnetism sensors 143 has directivity of magnetism detection sensitivity in a plurality of directions. For example, each magnetism sensor 143 is a so-called three-axis sensor including an X-axis sensor having a directivity of magnetism detection sensitivity in the X-axis direction, a Y-axis sensor having a directivity of magnetism detection sensitivity in the Y-axis direction, and a Z-axis sensor having a directivity of magnetism detection sensitivity in the Z-axis direction. The magnetism sensors 143 are examples of a composite magnetism sensor including a predetermined number of magnetism sensors having mutually different magnetism detection directions. The Z-axis sensor is an example of a first magnetism sensor. The X-axis sensor and the Y-axis sensor are examples of a second magnetism sensor.

The calculating part 161 estimates the position of the marker coil 110 by using only the magnetism data measured by the magnetism sensors whose magnetism detection directions are not parallel to the normal vector to the marker coil 110 among the signals processed by the signal processing part 160. That is, the calculating part 161 avoids using the magnetism data measured by the magnetism sensors whose magnetism detection directions are parallel to the normal vector to the marker coil 110 in the calculation for estimating the position of the marker coil 110. As a result, the position of the marker coil 110 can be accurately estimated as in the first embodiment.

The magnetism sensors whose magnetism detection directions are not parallel to the normal vector to the marker coil 110 are, for example, the X-axis sensors and the Y-axis sensors. The magnetism sensors whose magnetism detection directions are parallel to the normal vector to the marker coil 110 are, for example, the Z-axis sensors. The calculating part 161 may avoid using magnetism data measured by at least one of the magnetism sensors whose magnetism detection directions are parallel to the normal vector to the marker coil 110 in the calculation for estimating the position of the marker coil 110.

For example, the magnetism measuring apparatus 101 depicted in FIGS. 10A and 10B may be a measuring apparatus of measuring a magnetic field caused by neural activity in a living body such as a magnetospinograph or a magnetocardiograph. In this type of biomagnetism measuring apparatus, a plurality of magnetism sensors having detection sensitivities in the X-axis direction, detection sensitivities in the Y-axis direction, and detection sensitivities in the Z-axis direction are used. Each of the plurality of magnetism sensors may be a magnetism sensor having a single magnetism detection direction, or may be a composite magnetism sensor including a plurality of magnetism sensors having mutually different magnetism detection directions, respectively. This makes it easier to detect a signal source that moves in various directions as a portion of the living body acting as the signal source changes in association with nerve activity.

Also in the magnetism sensor array 141 of this type of biomagnetism measuring apparatus, the X-axis sensors, the Y-axis sensors, or both of the X-axis sensors and the Y-axis sensors, each of whose magnetism detection directions is not parallel to the normal vector to the marker coil 110, are used in calculation to estimate the position of the marker coil 110. Thus, the position of the marker coil 110 can be accurately estimated, and the biomagnetic field can be accurately measured by the biomagnetism measuring apparatus.

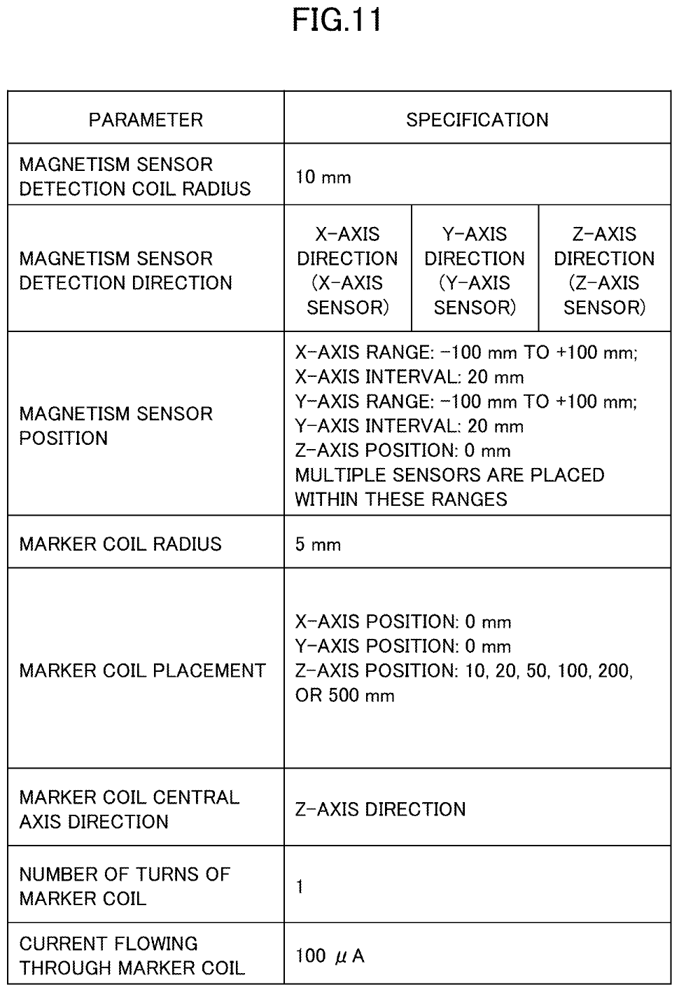

FIG. 11 is a diagram depicting an example of simulation conditions for estimating the position of the marker coil 110 by a magnetism measuring apparatus different from the magnetism measuring apparatus 101 of FIGS. 10A and 10B. Detailed description of the conditions same as or similar to the conditions in FIG. 7 will be omitted. The conditions (specifications) of the simulation are the same as or similar to the conditions (specifications) of the simulation of FIG. 7 except that each of the plurality of magnetism sensors 143 included the X-axis sensor, the Y-axis sensor, and the Z-axis sensor.

In the simulation, for example, instead of actually measuring magnetism using the X-axis sensors, the Y-axis sensors, and the Z-axis sensors of the plurality of magnetism sensors 143, the magnetic fields that would be generated according to the Biot-Savart law were calculated and were used to estimate the position of the marker coil 110. In the simulation, the position estimation errors were calculated for the case where only the X-axis sensors were used, the case where only the Y-axis sensors were used, and the case where only the Z-axis sensors were used.

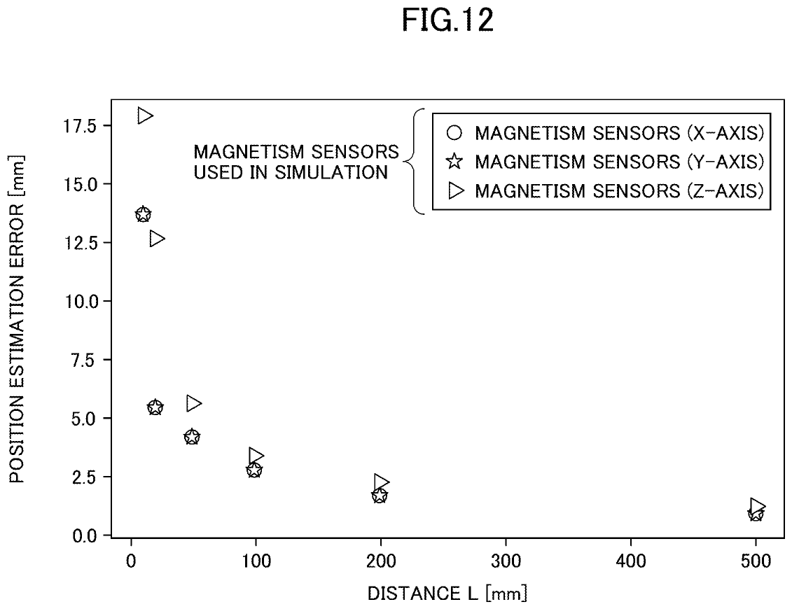

FIG. 12 is an explanatory diagram depicting an example of the estimation errors of the position of the marker coil 110 obtained using the simulation conditions depicted in FIG. 11. The horizontal axis and the vertical axis of the graph are the same as the horizontal axis and the vertical axis of FIG. 9, respectively. The circles in the graph represent the position estimation errors calculated for the case where only the X-axis sensors were used. The stars in the graph represent the position estimation errors calculated for the case where only the Y-axis sensors were used. The triangles in the graph represent the position estimation errors calculated for the case where only the Z-axis sensors were used.

As can be seen from FIG. 12, the position estimation errors calculated for the case where the X-axis sensors were used are substantially equal to the position estimation errors calculated for the case where the Y-axis sensors were used, and the position estimation errors for the case where the Z-axis sensors were used are greater than the position estimation errors for the case where the X-axis sensors were used and are greater than the case where the Y-axis sensors were used. Further, the smaller the distance L between the magnetism sensor 143 and the marker coil 110 is, the greater the difference between the position estimation error calculated for the case where the Z-axis sensors were used and the position estimation errors calculated for the case where the X-axis sensors or the Y-axis sensors were used is.

In other words, the errors in the estimated positions (position estimation errors) of the marker coil 110 calculated by the calculating part 161 can be made smaller and the position of the marker coil 110 can be estimated with higher accuracy for the case where the X-axis sensors or the Y-axis sensors are used than the case where the Z-axis sensors are used. Also in the second embodiment, the calculating part 161 may avoid using, in the calculation for estimating the position of the marker coil 110, the magnetism data measured by the magnetism sensors 143, each of whose distances from the marker coil 110 is equal to or less than 50 mm (first distance).

Thus, also in the second embodiment, the same advantageous effects as the advantageous effects of the first embodiment can be obtained. For example, the calculating part 161 avoids using the magnetism data measured by the magnetism sensors, each of whose magnetism detection direction is parallel to the normal vector to the marker coil 110, in the calculation for estimating the position of the marker coil 110. Alternatively, the calculating part 161 avoids using the magnetism data measured by the magnetism sensors 143, each of whose distance from the marker coil 110 is equal to or less than 50 mm (first distance), for example, in the calculation for estimating the position of the marker coil 110. Thus, the position of the marker coil 110 can be accurately estimated even using the magnetism sensors 143 having the plurality of magnetism detection directions, such as the three-axis sensors.

### Third Embodiment

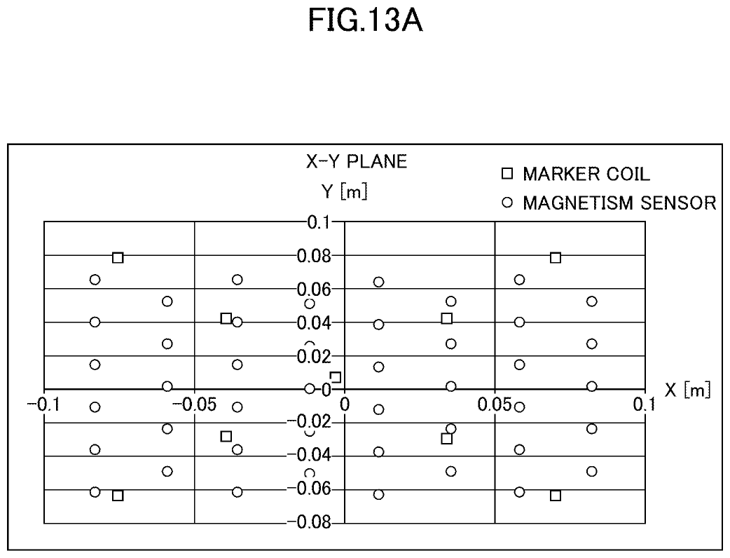

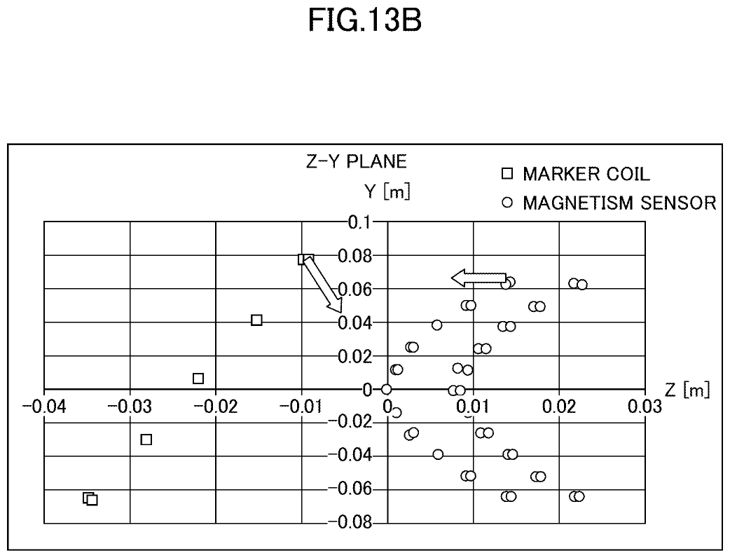

FIGS. 13A and 13B are explanatory diagrams depicting an example of positional relationships between each magnetism sensor of the magnetism sensor array and marker coils in a magnetism measuring apparatus according to a third embodiment. The plurality of marker coils are fixed on a flat plate. The marker coils fixed on the flat plate are used, for example, to confirm that the magnetism measuring apparatus is operating normally. The measured distances between the marker coils on the flat plate are compared with position estimation results of the marker coils. The position estimation results of the marker coils are calculated based on the magnetic field data that is measured by the magnetism measuring apparatus when the marker coils are excited. From the comparison, it can be determined whether the position estimation results are correct. Then, in a case where it is thus determined that the position estimation results are correct, it can be confirmed that the magnetism measuring apparatus is operating normally.

In the plurality of magnetism sensors of the magnetism sensor array depicted in FIGS. 13A and 13B, the positions of the extending ends of the magnetism sensors that are placed side by side in the Y direction are arranged in such a manner that the Z coordinates increase as the absolute values of the Y coordinates increase, and the extending ends of the magnetism sensors that are placed side by side in the Y direction draw a curve. By arranging the magnetism sensors in such a manner that the extending ends of the magnetism sensors placed side by side in the Y direction draw a curve, a distance (gap) between a measuring target and the magnetism sensors can be reduced for a case where the measuring target has a curved surface such as a neck of a human body, for example. The signal-to-noise ratio (S/N) can be improved by thus reducing the distance between the measurement target and the magnetism sensors. This is because the magnetic field attenuates with the third power of the distance. That is, the degree of attenuation decreases as the distance between the measuring target and the magnetism sensor decreases; therefore, the signal intensity increases and the S/N can be relatively improved.

In this regard, when the magnetic fields generated from the marker coils fixed on the flat plate are measured by the sensor array in which the magnetism sensors are placed side by side to draw a curve, it can be seen that some positional relationships result such that calculation errors in the magnetic fields increase depending on the marker coils. Therefore, before using the third embodiment, the user of the magnetism measuring apparatus would need to pay attention to place the flat plate in such a manner that the positional relationships that would result in the increase in the measurement errors of the magnetic fields could be avoided.

According to the third embodiment, magnetism sensors of the positional relationships that would result in the increase in the calculation errors of the magnetic fields can be avoided being used as the calculation bases. Therefore, the user does not need to pay special attention when arranging the flat plate to which the marker coils are fixed, and can more easily confirm that the magnetism measuring apparatus is operating normally. Note that the number and arrangement of the magnetism sensors and the number and arrangement of the marker coils depicted in FIGS. 13A and 13B are merely examples; the number and arrangement of the magnetism sensors and the number and arrangement of the marker coils are not limited to these numbers and arrangements.

In the Z-Y plane depicted in FIG. 13B, an arrow whose base end is at one of the marker coils indicates an example of a normal vector to the marker coil. In the Z-Y plane depicted in FIG. 13B, an arrow whose base end is at one of the magnetism sensors indicates an example of a normal vector to the sensor coil of the magnetism sensor. The plurality of marker coils have the mutually different cosine similarities with respect to each of magnetism sensors and have the mutually different distances to each of magnetism sensors. Therefore, one or more of the marker coils may have great calculation errors of the magnetic fields due to their positional relationships with respect to the magnetism sensors, and the estimation accuracies may vary among the plurality of marker coils. According to the third embodiment, the influences of the calculation errors of the magnetic fields can be reduced, and therefore, it is possible to reduce the variation in the estimation accuracies among the plurality of marker coils.

Also in the third embodiment, the same advantageous effects as the advantageous effects of the first embodiment and the second embodiment can be obtained. Furthermore, in the third embodiment, for example, even in the case where the plurality of marker coils are fixed on the flat plate that faces the magnetism sensor array and there are one or more of the marker coils having the positional relationships that would result in the increase in the calculation errors of the magnetic fields, the one or more of the magnetism sensors having the positional relationships that would result in the increase in the calculation errors of the magnetic fields can be avoided being used as the calculation bases. Therefore, the user of the magnetism measuring apparatus does not need to pay special attention when placing the flat plate, and can more easily confirm that the magnetism measuring apparatus is performing normally. Further, according to the third embodiment, the influences of the calculation errors of the magnetic fields can be reduced, and therefore, it is possible to reduce the variation in the estimation accuracies among the plurality of marker coils.

Although the magnetism measuring apparatuses, magnetism measurement processing apparatuses, and methods for controlling magnetism measurement processing apparatuses have been described with reference to the embodiments, the present invention is not limited to these embodiments, and various variations and improvements can be made within the scope of the present invention appropriately in accordance with particular application modes.

### RELATED ART DOCUMENTS

### Patent Documents

[Patent Document 1] Japanese Patent Application Publication No. 2016-106686

[Patent Document 2] Japanese Patent Application Publication No. 2017-217457

### Non Patent Documents

[Non-Patent Document 1] “Magnetospinography visualizes electrophysiological activity in the cervical spinal cord” Scientific Reports 7, Article number: 2192 (2017)

The contents of Non-Patent Document 1 are incorporated herein by reference in their entirety.

[Non-Patent Document 2] Daisuke Oyama, Yoshiaki Adachi, Masanori Higuchi, Jun Kawai, Masakazu Miyamoto, Koichiro Kobayashi, and Gen Uehara, “Real-time Head Localization System for Magnetoencephalography”, Journal of the Magnetics Society of Japan, Vol. 36, pp. 345-351, 2012

The contents of Non-Patent Document 2 are incorporated herein by reference in their entirety.

[Non-patent Document 3] Y. Adachi, Y. Mizuhara, and Y. Terazono, “Evaluation of Directional Dependence of Sensitivity for Room-Temperature Magnetic Flux Sensors With Wide Sensitivity Region,” in IEEE Transactions on Magnetics, vol. 57, no. 2, pp. 1-5, February 2021, Art no. 4000105, doi: 10.1109/TMAG. 2020.3008912

The contents of Non-Patent Document 3 are incorporated herein by reference in their entirety.

What is claimed is

1. A magnetism measuring apparatus comprising:
a plurality of magnetism sensors having magnetism sensitivities with respect to respective single directions;
a coil configured to generate a magnetic field in response to supply of a current; and
a processor configured to use a predetermined number of magnetism sensors from among the plurality of magnetism sensors to estimate a relative position between the coil and the predetermined number of magnetism sensors based on magnetic fields detected by the predetermined number of magnetism sensors,
wherein
the processor is configured to avoid using, in calculation for estimating the relative position, magnetism data measured by at least one of the plurality of magnetism sensors, the at least one of the plurality of magnetism sensors having an absolute value of a cosine similarity that is greater than or equal to a predetermined threshold, the cosine similarity being between a normal vector to the coil and a direction in which the at least one of the plurality of magnetism sensors has a magnetism sensitivity.

2. The magnetism measuring apparatus according to claim 1,
wherein
the processor is further configured to avoid using magnetism data measured by at least one of the plurality of magnetism sensors having an absolute value of the cosine similarity of “1” in the calculation for estimating the relative position.

3. The magnetism measuring apparatus according to claim 2,
wherein
the processor is further configured to avoid using magnetism data measured by a magnetism sensor of the plurality of magnetism sensors facing the coil in the calculation for estimating the relative position.

4. The magnetism measuring apparatus according to claim 1,
wherein
the processor is further configured to avoid using magnetism data measured by a magnetism sensor of the plurality of magnetism sensors having a linear distance to the coil smaller than or equal to a predetermined distance in the calculation for estimating the relative position.

5. The magnetism measuring apparatus according to claim 1, comprising:
a plurality of composite magnetism sensors each including a corresponding predetermined number of magnetism sensors from among the plurality of magnetism sensors,
wherein
each of the plurality of composite magnetism sensors includes a first magnetism sensor having a magnetism sensitivity in a direction parallel to the normal vector to the coil and one or more second magnetism sensors having magnetism sensitivities in respective directions not parallel to the normal vector to the coil.

6. The magnetism measuring apparatus according to claim 1,
wherein
the processor is further configured to
control operations of the plurality of magnetism sensors, and
avoid a detecting operation of the at least one of the plurality of magnetism sensors, use of the magnetism data of the at least one of the plurality of magnetism sensor being avoided in the calculation for estimating the relative position.

7. A magnetism measurement processing apparatus comprising
a processor configured to process magnetism data measured by a plurality of magnetism sensors having magnetism sensitivities with respect to respective single directions,
wherein
the processor is further configured to
use a predetermined number of magnetism sensors from among the plurality of magnetism sensors to estimate a relative position between the coil and the predetermined number of magnetism sensors based on magnetic fields detected by the predetermined number of magnetism sensors; and
avoid using, in calculation for estimating the relative position, magnetism data measured by at least one of the plurality of magnetism sensors, the at least one of the plurality of magnetism sensors having an absolute value of a cosine similarity that is greater than or equal to a predetermined threshold, the cosine similarity being between a normal vector to the coil and a direction in which the at least one of the plurality of magnetism sensors has a magnetism sensitivity.

8. A method for controlling a magnetism measurement processing apparatus, the magnetism measurement processing apparatus comprising a processor configured to process magnetism data measured by a plurality of magnetism sensors having magnetism sensitivities with respect to respective single directions, the method comprising:
using a predetermined number of magnetism sensors from among the plurality of magnetism sensors to estimate by the processor a relative position between the coil and the predetermined number of magnetism sensors based on magnetic fields detected by the predetermined number of magnetism sensors; and
avoiding, by the processor, using in calculation for estimating the relative position, magnetism data measured by at least one of the plurality of magnetism sensors, the at least one of the plurality of magnetism sensors having an absolute value of a cosine similarity that is greater than or equal to a predetermined threshold, the cosine similarity being between a normal vector to the coil and a direction in which the at least one of the plurality of magnetism sensors has a magnetism sensitivity.
